(19) United States
(12) Patent Application Publication
(10) Pub. No.: US 2026/0272303 A1
(43) Pub. Date: Sep. 17, 2026

(54) QUANTITATIVE MEASUREMENT OF THE PERIVASCULAR SPACE FOR CNS AND BRAIN DISORDERS

(71) Applicant: Northeastern University, Boston, MA (US)

(72) Inventors: Craig Ferris, Holden, MA (US); Ju Qiao, Boston, MA (US); Praveen Kulkarni, East Walpole, MA (US); Codi Gharagouzloo, Carlisle, PA (US)

(21) Appl. No.: 19/670,431

(22) Filed: May 7, 2026

Related U.S. Application Data
(63) Continuation of application No. 17/425,123, filed on Jan. 27, 2020, now Pat. No. 12,642,433.
(60) Provisional application No. 62/796,925, filed on Jan. 25, 2019.

(51) Int. Cl. A61B 5/00; A61B 5/055; A61K 49/06; G01R 33/56; G01R 33/561

(52) U.S. Cl. CPC A61B 5/0042; A61B 5/055; A61K 49/06; G01R 33/5601; G01R 33/5617

(57) ABSTRACT

Disclosed are methods for quantification of a perivascular space in a subject's central nervous system. The methods include administering contrast agent into cerebrospinal fluid of the subject; performing quantitative ultra-short time-to-echo contrast-enhanced magnetic resonance imaging (QUTE-CE MRI) on a region of interest of the subject's brain; and determining presence of the contrast agent in the perivascular space within the region of interest.

### RELATED APPLICATIONS

This application is a continuation of U.S. application Ser. No. 17/425,123, filed Jul. 22, 2021; which is a U.S. National Stage Application of International Application No. PCT/US20/15223, filed Jan. 27, 2020; which claims the benefit of priority to U.S. provisional patent application Ser. No. 62/796,925, filed Jan. 25, 2019.

### BACKGROUND

Previous efforts to use magnetic resonance imaging (MRI) to produce reproducible biomarkers for mild traumatic brain injury (TBI) have proven ineffective. In almost all cases, increases in perivascular space are reported, but no reliable imaging method existed to quantify the volume spatially. An increased volume of the perivascular space is also noted for all neurodegenerative diseases, such as Alzheimer's Disease (AD) and Parkinson's Disease (PD), and is also linked to glymphatic flow and clearance of toxins indicated.

The volume of the perivascular space is a unique biomarker because it is found around arterioles and venules, but not capillaries (e.g. spinal cord (Lam et al. 2017)). A large body of evidence indicates that perivascular spaces represent the major route for fluid inflow from the subarachnoid space into the brain and spinal cord (Bilston et al. 2003; Iliff et al. 2012; Rennels et al. 1985) and are also the major pathway for solute clearance. Early electron microscopic studies described perivascular spaces in hamster and rat, however the anatomical details of their ultrastructure are not well understood.

There is a need for methods that allow quantification of the volume of perivascular space in a subject and thereby facilitate determining changes in the volume of perivascular space, which can be an indication of disease.

### SUMMARY

Methods for quantification of a perivascular space in a subject's brain are disclosed. The perivascular space is dilated in subjects with traumatic brain injury, brain and CNS disorders. Quantification of a perivascular space in a subject's brain according to the methods disclosed herein can, for example, facilitate understanding, diagnosis and monitoring of the progress of traumatic brain injury, and brain and CNS disorders.

One embodiment is a method for quantification of a perivascular space in a subject's central nervous system comprising: administering contrast agent into cerebrospinal fluid of the subject; performing quantitative ultra-short time-to-echo contrast-enhanced magnetic resonance imaging (QUTE-CE MRI) on a region of interest of the subject's brain; and determining presence of the contrast agent in the perivascular space within the region of interest.

In some embodiments, the region of interest is the subject's brain, and the presence of the contrast agent (CA) is determined in perivascular spaces of the subject's brain.

In some embodiments, performing QUTE-CE MRI comprises applying a magnetic field to region of interest; applying a radio frequency pulse sequence with a selected repetition time (TR) and flip angle (FA) to excite protons in the region of interest, wherein the TR is less than about 10 ms, and the FA ranges from about 10° to about 30°; measuring a response signal during relaxation of the protons at a selected time-to-echo (TE) with magnetic field gradients activated to provide a T1-weighted signal from the region of interest, wherein the TE is an ultra-short time-to-echo (UTE) less than about 300 μs; and generating an image of the region of interest.

In some embodiments, the acquired signal is representative of a concentration of the CA in the region of interest.

In some embodiments, the methods disclosed herein further comprise setting the time to echo (TE) to less than 30 μs.

In some embodiments, the TR is set to a value below about 5 ms, the TE is set to a UTE value and the FA is selected to be around the Ernst angle of the contrast-enhanced blood.

In some embodiments, the methods disclosed herein further comprise setting the TE to zero using a Zero TE (ZTE) sequence.

In some embodiments, the methods disclosed herein further comprise setting the repetition time (TR) to a value from about 2 to about 10 ms.

In some embodiments, the methods disclosed herein further comprise setting the flip angle to a value from about 10° to about 25°.

In some embodiments, QUTE-CE MRI is performed before presence of the contrast agent in the perivascular space and is subsequently performed after contrast agent is present in the perivascular space.

In some embodiments, the contrast agent comprises ferumoxytol and QUTE-CE MRI is performed before administration of the contrast agent.

In some embodiments, the methods described herein comprise determining the presence of contrast agent in the perivascular space at a plurality of times based on respective quantitative signal intensity obtained by the QUTE-CE MRI at these times in the perivascular space.

In some embodiments, the methods described herein comprise determining the presence of contrast agent in the perivascular space when respective quantitative signal intensity obtained by QUTE-CE MRI for the perivascular space is near or at maximum value.

In further aspects of these embodiments, ferumoxytol is administered at a dose of about 2 to about 14 mgFe/kg body weight.

In further aspects of these embodiments, ferumoxytol is administered at a dose of about 3 to about 4 mgFe/kg body weight.

In further aspects of these embodiments, ferumoxytol is administered at a dose of about 7-14 mg/kg bodyweight in animals or 2-7 mg/kg bodyweight in humans.

In some embodiments, the subject is a non-human mammal.

In some embodiments, the subject is a human.

In some embodiments, the subject has traumatic brain injury (TBI), type 2 diabetes, stroke, or a CNS disorder.

In some embodiments, the subject has a CNS disorder; and the CNS disorder is amyotrophic lateral sclerosis, frontotemporal dementia, Huntington's disease, Alzheimer's disease or related dementia, or Parkinson's disease.

In some embodiments, the subject has an early stage CNS disorder selected from amyotrophic lateral sclerosis, Huntington's disease, Alzheimer's disease or related dementia, and Parkinson's disease.

In some embodiments, the subject has minor cognitive impairment.

In some embodiments, the administering of contrast agent is intrathecal.

### BRIEF DESCRIPTION OF THE DRAWINGS

FIG. 1A shows a 3D UTE Precontrast maximum intensity projection (MIP) image.

FIG. 1B shows a 3D UTE postcontrast at 56 minutes postcontrast MIP image.

FIG. 2A shows 2D axial image slices of T1-weighted anatomy.

FIG. 2B shows 2D axial image slices of 3D UTE Precontrast. postcontrast.

FIG. 2C shows 2D axial image slices of 3D UTE postcontrast at 56 minutes

FIG. 3A shows a maximum intensity projection image after 7 minutes.

FIG. 3B shows a maximum intensity projection image after 14 minutes.

FIG. 3C shows a maximum intensity projection image after 21 minutes.

FIG. 3D shows a maximum intensity projection image after 28 minutes.

FIG. 3E shows a maximum intensity projection image after 35 minutes.

FIG. 3F shows a maximum intensity projection image after 42 minutes.

FIG. 3G shows a maximum intensity projection image after 49 minutes.

FIG. 3H shows a maximum intensity projection image after 56 minutes.

### DETAILED DESCRIPTION

The various concepts introduced above and discussed in greater detail below may be implemented in any of numerous ways, as the described concepts are not limited to any particular manner of implementation. Examples of specific implementations and applications are provided primarily for illustrative purposes.

Methods for quantification of the volume of perivascular spaces in a subject's CNS are disclosed. The methods are based on the introduction of contrast agent (CA) into a subject's cerebral spinal fluid and its flow into perivascular spaces. After injection of CA into the cerebrospinal fluid, the CA perfuses throughout the central nervous system (CNS) and enhances the contrast of the cerebrospinal fluid (CSF). The presence of CA in the perivascular space can be measured, for example, by using ultrashort time echo (UTE) pulse sequences, such as with the QUTE-CE method, to generate a quantitative signal intensity, which can be followed longitudinally from the time of CA injection, for example, to determine the time course of signal intensity in the perivascular space. The presence of CA in a perivascular space is determined by the kinetics of flow into and flow out of the perivascular space. It has been found that quantification of the perivascular space is most accurate when done at a time when CA has substantially filled the perivascular space but before significant flow of CA out of the perivascular space, which is associated with significant decrease in signal intensity obtained by the QUTE-CE method. Following the signal intensity spatially (e.g., on a voxel-wise basis) and longitudinally allows determination of an optimum time for quantification of the perivascular space. It has further been found that the flow of ferumoxytol into and through perivascular spaces depends on whether the subject is awake or anesthetized. Specifically, it has been found that quantification of perivascular space in awake rats typically is most accurate when done within 5 to 15 minutes of injection of ferumoxytol, and that longer times are required for anesthetized rats.

The methods described herein can be used to quantify the volume of the perivascular spaces (particularly, the brain's perivascular spaces) in a subject, but they can also more generally be used to determine the dynamics of CA perfusion throughout the CNS including into the perivascular spaces and to quantify the volume of CSF in parts other than the perivascular spaces or the volume of CSF throughout the brain or CNS (including perivascular spaces). Patterns of the ratio, or spatial volume, of intraventricular space to the gray and white matter can reflect important measures of brain or CNS disease progression.

The volume of CSF including perivascular spaces in the CNS or brain quantified with the methods described herein, can be compared to the respective volume of a control group of healthy subjects. If the quantified volume, for example, of the perivascular spaces of a given subject's brain, is significantly larger than that of the control group, this can be an indication of traumatic brain injury (including mild traumatic brain injury), a brain disorder and/or CNS disorder of the given subject. Further, quantifying the volume, for example, of the perivascular spaces of a given subject's brain over days, weeks, or years can allow following progression of a traumatic brain injury, brain disorder and/or CNS disorder, by comparing the volumes quantified at different times. For example, if the perivascular spaces are increasing over time, this can be an indication of worsening of a brain disorder or disease. Accordingly, the methods described herein can be used in methods of diagnosing or monitoring the progression of a traumatic brain injury, brain disorder or CNS disorder.

A further embodiment is a method for diagnosing or monitoring the progression of a traumatic brain injury, brain disorder or CNS disorder in a subject, comprising using any one of the methods described herein for quantifying volume of CSF in a part (e.g., in the brain's perivascular spaces) of the subject's CNS, and comparing the volume so quantified with a respective volume of one or more control subjects. In an aspect of this embodiment, the quantified volume is compared to the respective volume of the same subject measured at an earlier time, for example, at a time that the subject was healthy (i.e., did not have a disorder causing dilation of one or more perivascular spaces such as traumatic brain injury, brain or CNS disorder).

Quantitative ultra-short time-to-echo (QUTE-CE) MRI, as previously described in (Gharagouzloo et al., 2015), (Gharagouzloo et al., 2017) and (U.S. Patent Application Publication No. 2019/0246938, entitled “Quantitative Magnetic Resonance Imaging of the Vasculature”; incorporated by reference) can be used in the methods disclosed herein. QUTE-CE is a quantitative, ultrashort time to echo, contrast-enhanced magnetic resonance imaging technique. The technique can be used to accurately measure contrast agent concentration in the blood, to provide clear, high-definition angiograms, and to measure absolute quantities of cerebral blood volume on a voxel-by-voxel basis.

QUTE-CE MRI is a technique that allows producing a quantitative signal intensity for MRI. This quantitative signal intensity has been shown to be used to quantify contrast agent concentration in blood (Gharagouzloo et al., 2015) and quantify contrast-enhanced blood per voxel in the brain (Gharagouzloo et al., 2017).

In the underlying theory of QUTE-CE MRI, the intensity magnitude I_{M} of each voxel is a function of standard MRI parameters governed by the Spoiled Gradient Echo (SPGR) equation,

$${I}_{M} = K ρ \cdot {e}^{( - TE / {T}_{2} )} \cdot sin ( FA ) \frac{1 - {e}^{( - TR / {T}_{1} )}}{1 - {e}^{( - TR / {T}_{1} )} \cdot cos ( FA )} , \qquad ( 1 )$$

where TE is the time-to-echo, TR is the repetition time, and FA is the flip angle. TE, TR, and FA are user-defined image acquisition parameters. T₁ and T₂ are relaxation time constants dependent on the local environment of each voxel, which is mutable via CAs, and dependent on the magnetic field strength. K is a constant determined by the properties of the receive coil and p is the proton density of the medium. TE is typically chosen to be <100 μs to eliminate susceptibility-induced signal modifications. The choice of a very low TR (<5 ms) with a 3D volume excitation pulse minimizes effects from the extravascular water exchange and eliminates signal enhancement from blood flow within the cranial space. This can be achieved with either hard pulse or slab-select pulse. Setting the FA at the Ernst angle maximizes the T1-enhanced signal and minimizes sensitivity to small perturbations in FA.

In some embodiments, performing QUTE-CE MRI comprises applying a magnetic field to region of interest; applying a radio frequency pulse sequence at a selected TR and at a magnetic field gradient to provide a selected FA to excite protons in the region of interest, wherein the TR is less than about 10 ms, and the FA ranges from about 10° to about 30°; measuring a response signal during relaxation of the protons at a selected TE to acquire a T₁-weighted signal from the region of interest, wherein the time to echo is an ultra-short time to echo less than about 300 μs; and generating an image of the region of interest.

In some embodiments, the acquired signal is representative of a concentration of the CA in the region of interest.

In some embodiments, the disclosed methods further comprise setting the TE to less than about 30 μs.

In some embodiments, the disclosed methods further comprise setting the TR to a value from about 1 to about 10 ms.

In some embodiments, the disclosed methods further comprise setting the flip angle to a value from about 10° to about 25°.

In some embodiments, QUTE-CE MRI is performed using a TE of about 13 μs, a TR of about 4 ms and an FA of about 20° with a high radio frequency (RF) pulse bandwidth of 200 kHz. This results in a pulse duration of 6.4 μs which is short compared to the T₂ of the approximate ferumoxytol concentration (Gharagouzloo et al., 2017). Choosing a pulse duration which is short compared to the T₂ of the approximate ferumoxytol concentration minimizes signal blur and reduce the probability for a curved trajectory of the magnetization vector M_{z} (Gharagouzloo et al., 2017).

QUTE-CE MRI can be performed with commercially available magnetic resonance imaging devices, as they are found, for example, in clinical, hospital and medical laboratory settings. QUTE-CE MRI is a technique that produces a quantitative signal intensity for MRI. This quantitative signal intensity has been shown to be used to quantify contrast agent concentration in blood (Gharagouzloo et al., 2015) and quantify contrast-enhanced blood per voxel in the brain (Gharagouzloo et al., 2017).

The methods disclosed herein, include performing the QUTE-CE MRI method on a region of interest of the subject's CNS. For example, a region of interest can be a part of the subject's brain or the entirety of the subject's brain.

After administration of CA (e.g., intrathecal administration of ferumoxytol) to the subject, it takes some time for CA to flow into perivascular spaces. Accordingly, typically, a pre-contrast image of the region of interest is determined, and, subsequently, one or more images are acquired to determine a baseline (i.e., before ferumoxytol has been able to localize in the perivascular spaces, subsequently, one more images are acquired to determine ferumoxytol concentration in the perivascular spaces of the region of interest.

In some embodiments, QUTE-CE MRI is performed before injection of CA, e.g., ferumoxytol.

Further information regarding performing the QUTE-CE MRI method, including preparing a brain atlas containing a plurality of anatomical and functional regions, obtaining a high-resolution anatomical data set of the region of interest, and image processing are provided in (Gharagouzloo et al., 2017). The methods described herein can include imaging the region of interest with voxels registered to a brain atlas.

The methods disclosed herein include administration of a CA. Suitable CA are paramagnetic and flow into the perivascular space. Typically, the CA is ferumoxytol. Ferumoxytol is an ultra-small superparamagnetic iron oxide nanoparticle (SPION) with a dextran coating. Since the size exceeds the cutoff (~6 nm) for glomerular filtration, ferumoxytol is not cleared by the kidney, and instead is an excellent blood pool CA with a long intravascular half-life of about 15 h (Bremerich et al., 2007). Ferumoxytol is approved for iron-deficiency anemia. Ferumoxytol has been found to be particularly useful as contrast agent in the methods described herein because of its localization to the perivascular space with a prolonged residence time (i.e., favorable pharmacokinetics). This facilitates longitudinal and highly accurate measurements.

In some embodiments, the CA is not cleared by the kidney of the subject. In other embodiments, the CA has a size which enables kidney filtration.

In the methods described herein, contrast agent is injected into the cerebrospinal fluid. Typically, the contrast agent is ferumoxytol. Commercially available Feraheme® injection can be used, which is a sterile aqueous colloidal product that is formulated with mannitol. It is a black to reddish brown liquid, and is commercially available in single-dose 17 mL vials containing 510 mg of elemental iron. Each mL of the sterile colloidal solution of Feraheme® Injection contains 30 mg of elemental iron, 30 mg polyglucose sorbitol carboxymethylether, and 44 mg of mannitol. The formulation is isotonic with an osmolality of 270-330 mOsm/kg. The product contains no preservatives, and has a pH of 6 to 8.

In some embodiments, ferumoxytol is administered at a dose of about 1 mgFe/kg body weight to about 8 mgFe/kg body weight, about 2 mgFe/kg body weight to about 7 mgFe/kg body weight, about 2 mgFe/kg body weight to about 6 mgFe/kg body weight, about 2 mgFe/kg body weight to about 4 mgFe/kg body weight, or about 4 mgFe/kg body weight.

As used herein, “body weight” refers to the body weight of the subject.

In some embodiments, determining presence of contrast agent (e.g., ferumoxytol) includes assigning signal intensity acquired using QUTE-CE MRI on a voxel basis to perivascular space within the region of interest. Further, determining presence of contrast agent (e.g., ferumoxytol) can include calculating the voxel wise change of the intensity of the acquired signal from a first acquired signal at a time before contrast agent is present in the one or more perivascular spaces within the region of interest (typically, at a time before administration of contrast agent) to a subsequently acquired signal after contrast agent administration. When signal is acquired repeatedly (e.g., continuously or periodically) from the time that contrast agent (e.g., ferumoxytol) is administered, the methods disclosed herein also allow following the dynamics of contrast agent perfusion in the region of interest.

The methods disclose herein can be used to quantify one or more perivascular spaces in subjects in need thereof. As used herein, a “subject in need thereof”, refers to a subject who is at risk to have, is suspected to have, and/or has a traumatic brain injury, brain disorder or CNS disorder.

In embodiments, the subject in need thereof is a subject who has or had a traumatic brain injury, has had or has a stroke, has type 2 diabetes, or has a central nervous system (CNS) disorder, for example, amyotrophic lateral sclerosis, frontotemporal dementia, Huntington's disease, Alzheimer's disease or related dementia, Parkinson's disease, and mental illnesses, such as schizophrenia and depression.

In embodiments, the subject in need thereof is a subject who has an early stage CNS disorder. Examples of early stage CNS disorders include, but are not limited to, early stage amyotrophic lateral sclerosis, early stage Huntington's disease, early stage Alzheimer's disease or related dementia, early stage Parkinson's disease, early stage frontotemporal dementia, or an early stage minor cognitive impairment.

As used herein, “subject” refers to a mammal, including, but not limited to, a human or non-human mammal, such as a bovine, equine, canine, ovine, rodent, or feline.

Disease models in small animals, including transgenic models of multiple CNS disorders, such as Huntington's Disease, Alzheimer's disease and Parkinson's disease, can also be quantified with the technology.

The Alzheimer's Association study published a study on the economic benefit early AD detection. The model included the entire U.S. population in 2018, with early detection measures beginning in 2020. Differences in expected costs come from two primary sources: “1) there is a ‘spike’ in costs during the period immediately before and after diagnosis, and this spike is smaller when diagnosis is made during the MCI stage, and 2) medical and long-term care costs are lower in people with diagnosed and managed MCI and dementia than in people with unmanaged MCI and dementia.”

With 80M people in the age range of 45-64, a 1% diagnostic check per year from this population would yield 800K scans, with a total gross cost of $1.6B-$2.8B. If all AD cases with mixed vascular pathology are diagnosed early the cost benefits for the current US population would be as much as $3.16 trillion (calculated as 40% of the potential $7.9 trillion in savings for all AD), or simply $12.72-$92.56 billion per year from 2025-2050 (see Graph); specifically, these cost savings would be to Medicare (47%), Medicaid (32%) and other insurers (20%). Based on the potential financial benefits to insurers, we project that insurance companies will cover the costs of these early diagnostic tests. With insurance companies willing to pay and 80M people standing to benefit, we expect health facilities and MRI manufacturers to integrate and use the technology.

### Further Definitions

As used herein, the singular forms “a,” “an” and “the” include plural referents unless the content clearly dictates otherwise. For example, reference to “a cell” includes a combination of two or more cells, and the like.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

As used herein, “intrathecal administration of a contrast agent” or “administering intrathecally a contrast agent” refers to a route of administration of contrast agent via an injection into the subject's spinal canal or subarachnoid space so that the contrast agent reaches the cerebrospinal fluid (CSF).

As used herein, “perivascular spaces (PVSs)” are pial-lined, fluid-filled structures found in characteristic locations throughout the brain. They are also known as Virchow-Robin spaces.

### EXAMPLES

The present examples are non-limiting implementations of the present technology.

Quantitative vascular mapping of the rat brain CSF starts with acquisition of pre- and post-ferumoxytol scans. A 3D UTE sequence with optimized parameters for perivascular contrast and quantification are utilized. Field corrections for coil sensitivity (B1−) and flip-angle distribution (B1+) are applied along with motion correction between the pre- and post-contrast images. A voxel-wise calculation for the quantitative CSF (qCSF) is performed to produce the qCSF maps after using a two-volume CSF and blood/tissue model with knowledge of CSF intensity obtained from large ventricles. For the rat models described here, voxels are distributed into an anatomically segmented atlas with 173 regions for quantitative analysis of the whole brain. Statistically significant abnormalities are found by comparing healthy rats to those of the relevant disease model or indication.

METHOD/PROCEDURE: Just prior to imaging, rats were anesthetized with 2-3% isoflurane. The scalp was incised and a burr hole made in the skull for implantation of sterile PE10 tubing (Braintree Scientific) aimed at the right lateral cerebroventricle using the stereotaxic coordinates: 1.0 mm posterior to the bregma, 2.0 mm lateral to the midline, and 4.0 mm in depth from dura. The tubing, ca 60 cm in length and prefilled with Ferumoxytol, was fixed in place with cyanoacrylic cement and connected to a 0.3 mL syringe needle filled with the contrast agent Ferumoxytol that could be positioned just outside the bore of the magnet. Ferumoxytol concentration for the second animal was 10 mg/ml Fe. Volume injected for each animal is 20 microliters. An anatomy imaging and a UTE imaging were taken before contrast administration. Time series of UTE imagings were taken immediately after ferumoxytol injection.

Scan parameters: TE/TR=0.01 ms/4 ms, FA=20 degrees, 3 cm isotropic field of view (FoV) with 166.7 micrometer istropic resolution, scan time 7 minutes.

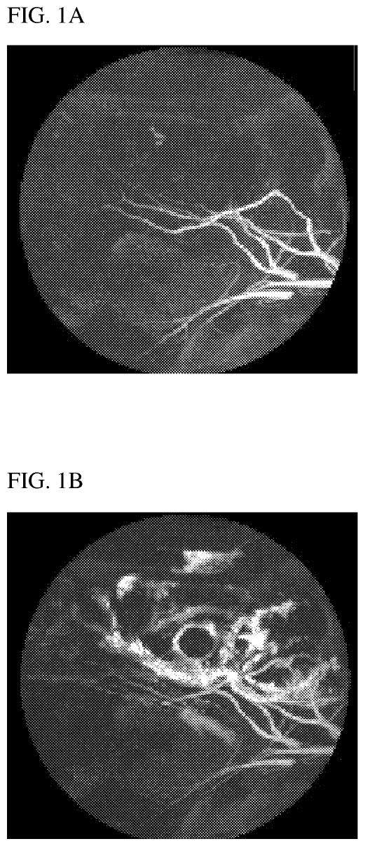

FIGS. 1A and 1B show (i) 3D UTE Precontrast maximum intensity projection (MIP) image, and (ii) 3D UTE postcontrast at 56 minutes postcontrast MIP image, respectively.

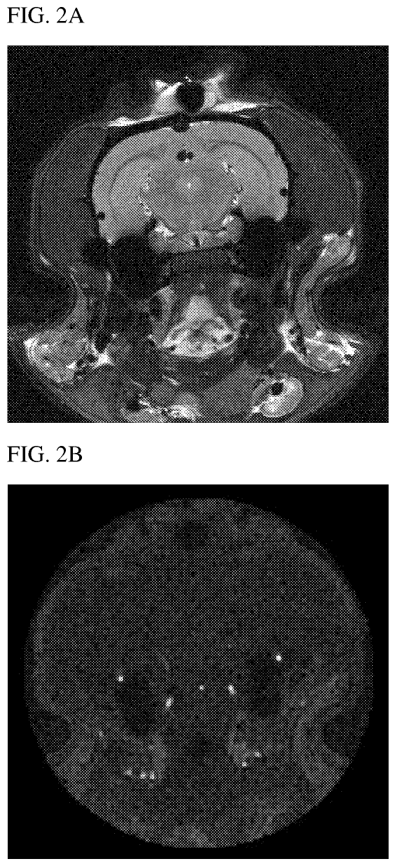

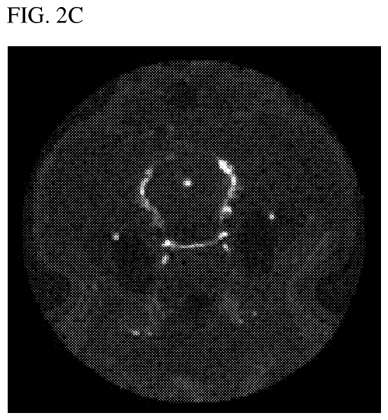

FIGS. 2A, 2B and 2C show 2D axial image slices of (i) T1-weighted anatomy, (ii) 3D UTE Precontrast, and (iii) 3D UTE postcontrast at 56 minutes postcontrast, respectively.

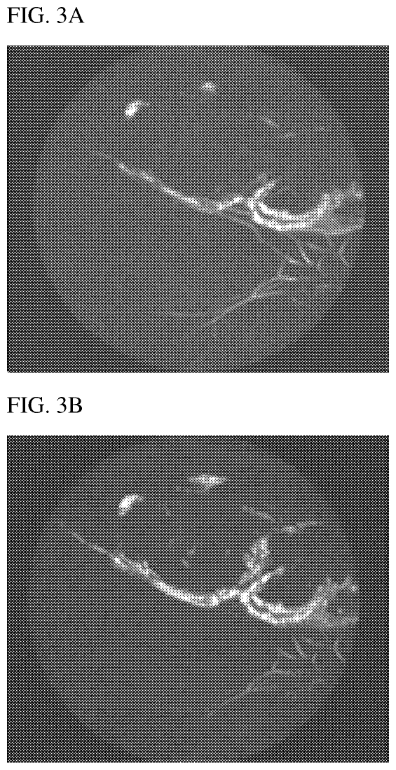

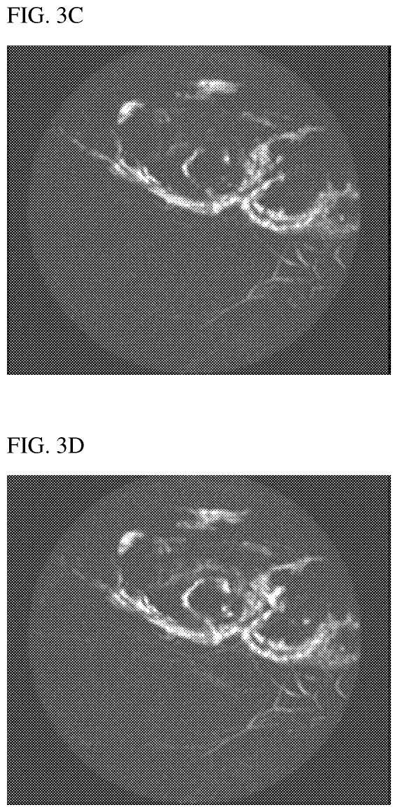

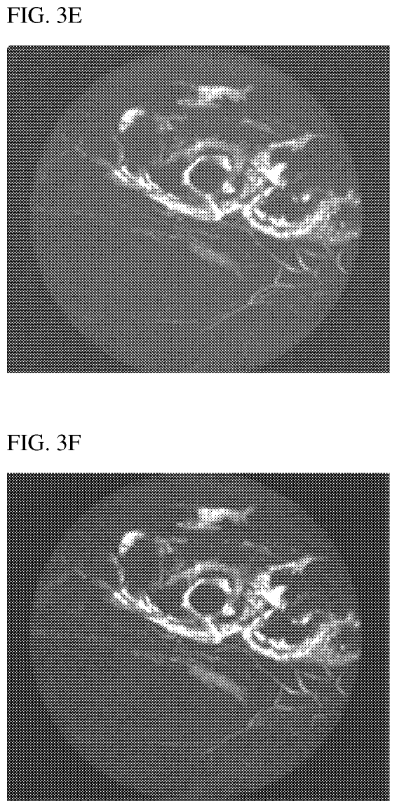

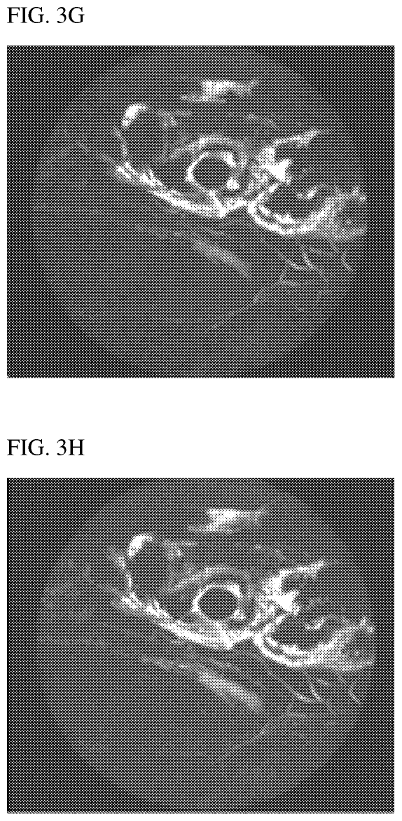

FIGS. 3A, 3B, 3C, 3D, 3E, 3F, 3G, and 3H show Maximum intensity projection images (MIPS) of the rat demonstrating time-course of glymphatic mixing using the method. All images are rendered by subtracting the first image without contrast, such that the time-course is clearly visualized. Images were run continuously with images ending after (a) 7 minutes, (b) 14 minutes, (c) 21 minutes, (d) 28 minutes, (e) 35 minutes, (f) 42 minutes, (g) 49 minutes, and (h) 56 minutes, respectively.

### REFERENCES CITED

Bilston, L. E., Fletcher, D. F., Brodbelt, A. R. & Stoodley, M. A. Arterial pulsation-driven cerebrospinal fluid flow in the perivascular space: A computational model. Comput. Methods Biomech. Biomed. Engin. (2003). doi: 10.1080/10255840310001606116.
Bremerich J, Bilecen D, Reimer P. MR angiography with blood pool contrast agents. European radiology 2007; 17(12): 3017-24.
Gharagouzloo C A, McMahon P N, Sridhar S. Quantitative contrast-enhanced MRI with superparamagnetic nanoparticles using ultrashort time-to-echo pulse sequences. Magn Reson Med 2015; 74(2): 431-41.
Gharagouzloo C A, Timms L, Qiao J, Fang Z, Nneji J, Pandya A, et al. Quantitative vascular neuroimaging of the rat brain using superparamagnetic nanoparticles: New insights on vascular organization and brain function. Neuro Image 2017; 163:24-33.
Iliff, J. J. et al. A paravascular pathway facilitates CSF flow through the brain parenchyma and the clearance of interstitial solutes, including amyloid beta. Sci. Transl. Med. (2012), 4(147), 1547ra111. doi: 10.1126/scitranslmed.3003748.
Lam, M. A. et al. The ultrastructure of spinal cord perivascular spaces: Implications for the circulation of cerebrospinal fluid. Sci. Rep. (2017). doi:10.1038/s41598-017-13455-4.
Rennels, M. L., Gregory, T. F., Blaumanis, O. R., Fujimoto, K. & Grady, P. A. Evidence for a ‘Paravascular’ fluid circulation in the mammalian central nervous system, provided by the rapid distribution of tracer protein throughout the brain from the subarachnoid space. Brain Res., 1985, 326(1), pp. 47-63.
U.S. Patent Application Publication No. 2019/0246938, entitled “Quantitative Magnetic Resonance Imaging of the Vasculature”; incorporated by reference.

The teachings of the documents cited herein are hereby incorporated by reference.

### EQUIVALENTS

The present technology (including present methods) is not limited to the particular embodiments described in this application, which are intended as individual illustrations of aspects of the present technology. Many modifications and variations of the present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, compounds, compositions, disease pathologies, or devices, which can, of course, vary. It is also to be understood that the terminology used herein is for the purpose of describing particular embodiments only, and is not intended to be limiting.

What is claimed is:

1. A method for quantification of a perivascular space in a subject's central nervous system comprising: administering contrast agent into cerebrospinal fluid of the subject; performing quantitative ultra-short time-to-echo contrast-enhanced magnetic resonance imaging (QUTE-CE MRI) on a region of interest of the subject's brain; and determining presence of the contrast agent in the perivascular space within the region of interest.

2. The method of claim 1, wherein the region of interest is the subject's brain and the presence of the contrast agent is determined in perivascular spaces of the subject's brain.

3. The method of claim 1 or 2, wherein performing QUTE-CE MRI comprises applying a magnetic field to the region of interest; applying a radio frequency pulse sequence with a selected repetition time (TR) and a selected flip angle (FA) to excite protons in the region of interest, wherein the repetition time is less than about 10 ms, and the flip angle ranges from about 10° to about 30°; measuring a response signal during relaxation of the protons at a selected time to echo (TE) with magnetic field gradients activated to provide a T₁-weighted signal from the region of interest, wherein the time-to-echo is an ultra-short time-to-echo, or Zero TE (ZTE), less than about 300 μs; and generating an image of the region of interest.

4. The method of claim 3, wherein the acquired signal is representative of a concentration of the contrast agent in the region of interest.

5. The method of any one of claims 2 to 4, further comprising setting the time to echo (TE) to less than about 30 μs.

6. The method of any one of claims 2 to 5, further comprising setting the repetition time to a value from about 2 to about 10 ms.

7. The method of any one of claims 2 to 6, further comprising setting the flip angle to a value from about 10° to about 25°.

8. The method of any one of claims 1 to 7, wherein QUTE-CE MRI is performed before presence of the contrast agent in the perivascular space and is subsequently performed after contrast agent is present in the perivascular space.

9. The method of claim 8, wherein the contrast agent comprises ferumoxytol and QUTE-CE MRI is performed before administration of the contrast agent.

10. The method of any one of claims 1 to 9, comprising determining the presence of contrast agent in the perivascular space at a plurality of times based on respective quantitative signal intensity obtained by the QUTE-CE MRI at these times in the perivascular space.

11. The method of any one of claims 1 to 10, further comprising determining the presence of contrast agent in the perivascular space when respective quantitative signal intensity obtained by QUTE-CE MRI for the perivascular space is near or at maximum value.

12. The method of any one of claims 1 to 11, wherein the contrast agent is ferumoxytol.

13. The method of claim 12, wherein ferumoxytol is administered at a dose of about 2 to about 14 mgFe/kg body weight.

14. The method of claim 12, wherein ferumoxytol is administered at a dose of about 3 to about 4 mgFe/kg body weight.

15. The method of any one of claims 1 to 14, wherein the subject is a non-human mammal.

16. The method of any one of claims 1 to 14, wherein the subject is a human.

17. The method of any one of claims 1 to 16, wherein the subject has traumatic brain injury, type 2 diabetes, stroke, or a CNS disorder.

18. The method of claim 17, wherein the subject has a CNS disorder; and the CNS disorder is amyotrophic lateral sclerosis, frontotemporal dementia, Huntington's disease, Alzheimer's disease or related dementia, or Parkinson's disease.

19. The method of any one of claims 1 to 18, wherein the subject has minor cognitive impairment.

20. The method of any one of claims 1 to 19, wherein the administering of contrast agent is intrathecal.
